(19) United States
(12) Patent Application Publication
(10) Pub. No.: US 2014/0378853 A1
(43) Pub. Date: Dec. 25, 2014

(54) Universal wearable limb band mounting, powering and providing an antenna for, diverse physiological sensors and monitors

(71) Applicants: George Anthony McKinney, San Diego, CA (US); Keith Mathis, San Diego, CA (US); David Nichols, San Diego, CA (US); Mukund Halthore, Thousand Oaks, CA (US)

(72) Inventors: George Anthony McKinney, San Diego, CA (US); Keith Mathis, San Diego, CA (US); David Nichols, San Diego, CA (US); Mukund Halthore, Thousand Oaks, CA (US)

(21) Appl. No.: 13/506,830

(22) Filed: May 17, 2012

Related U.S. Application Data
(60) Provisional application No. 61/549,589, filed on Oct. 20, 2011.

(51) Int. Cl. A61B 5/024; A61B 5/0245

(52) U.S. Cl. CPC A61B 5/02438; A61B 5/0245
USPC 600/509

(57) ABSTRACT

A band, or bracelet, is semi-permanently attachable to, and wearable about, a limb of the human body. The limb band is in the physical form of a familiar, and well-accepted, flexible plastic hospital-type wrist bracelet. As well as providing a physical platform to attach, and to mount, any of a large number of diverse portable electronic physiological, motion and like sensors of the human body and its condition, the limb band houses, and electrically connects, any of (1) a battery that is preferably rechargeable, (2) a solar cell for recharging the battery, (3) an antenna, and/or (4) a universal electronic signal connector (such as a miniature USB connector port). In this manner the limb band economically serves as a universal platform for mounting, supplying power and/or abetting wireless and wired communication to and from, wearable electronic sensors and monitors of humans and animals.

### REFERENCE TO RELATED APPLICATIONS

The present application is related to, and claims benefit of priority of, U.S. Provisional Patent application Ser. No. 61/549,589 filed Oct. 11, 2011, for a ““Detachable self-powering wrist band” or “A means for monitoring a patients location and vital signs”. The provisional patent application is in the name of the selfsame inventors of the present application.

### BACKGROUND OF THE INVENTION

1. Field of the Invention

The present invention generally concerns a universal band, or bracelet—of comfortable and familiar form like as to a disposable hospital identification bracelet—that is worn upon a limb of the human body to provide (1) a physical mounting, (2) a battery power supply, (3) solar cell recharging circuitry, and/or (4) a communications antenna to any of diverse electronic physiological sensors and monitors.

The present invention particularly concerns a universal wearable flexible band, or bracelet platform—including as is commonly semi-permanently snapped about the circumference of a limb of the human body—serving to (1) mount, (2) rechargeably power, and/or (3) abet communications with, any of diverse portable wearable electronic physiological sensors and monitors.

2. Background of the Invention

2.1 Limb and Wrist Bands Containing Batteries are Known

The present invention will be seen to concern a limb, or wrist, band, or a bracelet, containing, among other things, a battery. Wrist bands containing batteries are known.

Tokyo-based gadget maker Thanko—generally doing business as USB specialists—are producing circa 2011 a Wristband Battery [JP]; which is precisely what it sounds like and supposed to make life easier for fans of portable gadgets.

Thanko says the wristband, which houses a 1500 mAh lithium-ion battery, can be used for the PSP, the Nintendo DS Lite (not the original DS), all iPods of the third generation or newer (not the iPhone), and a number of Japanese cell phones (buyers will get five different adapters). Thanko says their battery will power a DS (on the highest brightness setting) for up to 12 hours and a PSP for up to 4.5 hours.

Although these power characteristics are suited for the intended uses, the bracelet of the present invention will be seen to use sustainable, and rechargeable components that—while producing less power than does the Thanko wristband—(1) are but a small fraction of the weight and size of the Thanko unit, that (2) are not only rechargeable but (in the most preferred embodiment of the invention) are actively recharged during use, and that (3) are intended and adapted to power micro-powered and microminiaturized sensors most commonly in a clinical application.

2.2 Miniaturized Accelerometers Usable with the Limb Band of the Present Invention

A Single-Axis, High-g, iMEMS® Accelerometer type ADXL193 from Analog Devices, Inc. is suitably used with the limb band of the present invention as a wearable device to sense acceleration, such as in a fall. “The ADXL193 is a low power, complete single-axis accelerometer with signal conditioned voltage outputs that are all on a single monolithic IC. This product measures acceleration with a full-scale range of ±120 g or ±250 g (minimum). It can also measure both dynamic acceleration (vibration) and static acceleration (gravity).

“The ADXL 193 is a fourth-generation surface micromachined iMEMS accelerometer from ADI with enhanced performance and lower cost. Designed for use in front and side impact airbag applications, this product also provides a complete cost-effective solution useful for a wide variety of other applications. The ADXL193 is temperature stable and accurate over the automotive temperature range, with a self-test feature that fully exercises all the mechanical and electrical elements of the sensor with a digital signal applied to a single pin. The ADXL 193 is available in a 5 mm×5 mm×2 mm, 8-terminal ceramic LCC package.”

Yet another accelerometer suitable for use with the limb band of the present invention is the MMA8450Q 3-Axis, 8-bit/12-bit Digital Accelerometer from Freescale Semiconductor, Inc. This device [the MMA8450Q] “is a smart low-power, three-axis, capacitive micromachined accelerometer featuring 12 bits of resolution. This accelerometer is packed with embedded functions with flexible user programmable options, configurable to two interrupt pins. Embedded interrupt functions allow for overall power savings relieving the host processor from continuously polling data. The MMA8450Q's Embedded FIFO buffer can be configured to log up to 32 samples of X, Y and Z-axis 12-bit (or 8-bit for faster download) data. The FIFO enables a more efficient analysis of gestures and user programmable algorithms, ensuring no loss of data on a shared I2C bus, and enables system level power saving (up to 96% of the total power consumption savings) by allowing the applications processor to sleep while data is logged. There is access to both low pass filtered data as well as high pass filtered data, which minimizes the data analysis required for jolt detection and faster transitions. The MMA8450Q has user selectable full scales of ±2 g/±4 g/±8 g. The device can be configured to generate inertial wake-up interrupt signals from any combination of the configurable embedded functions allowing the MMA8450Q to monitor events and remain in a low power mode during periods of inactivity.”

2.3 A Miniaturized Blood Pressure Sensor Usable with the Limb Band of the Present Invention

The SphygmoCor® CP System from Atcor Medical is suitably used with the limb band of the present invention as a wearable sensor for sensing in real time the blood pressure of a patient wearer to the limb band. The system is claimed by its manufacturer to be “the gold standard in noninvasive central blood pressure assessment. It has been featured in hundreds of published studies, and is used in leading medical centers and in pharmaceutical clinical trials worldwide.

“The SphygmoCor CP system acquires the patient's radial pulse waveform through a measurement taken at the wrist, derives the blood pressure waveform at the ascending aorta and reports vital central blood pressure data.”

Yet another wearable blood pressure sensor system is described in United States Patent Application publication no. 20050054907 for a HIGHLY PORTABLE AND WEARABLE BLOOD ANALYTE MEASUREMENT SYSTEM to Joseph Page, et al. The patent application, abandoned, describes “non-invasive wearable systems for continuous measurement of blood glucose concentrations help diabetics maintain best awareness and control. A wearable article such as a wristwatch includes elements integrated therewith to provide for biometric measurements. Specifically, both optical and acoustic transducers are arranged within an article such that they are coupled to tissue in a manner which permits blood analytes measurements to be made. In best versions, a quantum cascade laser is arranged with crystalline acoustic detectors in a photoacoustic effect measurement scheme. Laser pulses stimulate special vibrational states of glucose molecules to produce an acoustic return signal to be received at a piezoelectric detector. A wristwatch case may include a back member which supports arrangements and coupling between the back of the watch, elements contained therein, and tissue in contact with the device.”

### SUMMARY OF THE INVENTION

The present invention contemplates a band, or bracelet, that is semi-permanently attachable to, and wearable about, a limb of the human body. The limb band is preferably in the physical form of a familiar, and well-accepted, flexible plastic hospital-type wrist bracelet. As well as providing a physical platform to attach, and to mount, any of a large number of diverse portable electronic physiological, motion and like sensors of the human body and its condition, the limb band houses, and electrically connects, any of (1) a battery that is preferably rechargeable, (2) a solar cell for recharging the battery, (3) an antenna, and/or (4) a universal electronic signal connector (such as a miniature USB connector port). In this manner the limb band economically serves as a universal platform for mounting, supplying power and/or abetting wireless and wired communication to and from, wearable electronic sensors and monitors of humans and animals.

The limb band electrical sensor platform of the present invention is preferably (1) water resistant and impervious to normal use and conditions encountered upon a limb of the human body, (2) adjustable in fit, (3) lightweight, (4) flexible including in both its battery and solar cell components, (5) removable and either reusable or discardable, and (6) both mechanically and electrically quick-connecting to both physically mount and electrically connect an electronic sensor or monitor.

The universal limb band universal sensor platform of the present invention intentionally resembles a normal hospital identification bracelet and, indeed, can bear the same indicia as such a bracelet—thereby serving a purpose as an identification bracelet is well as a platform for a patient sensor(s) or monitor(s). Microminiaturized micro-powered sensors and monitors that most commonly have a medical (or veterinary) purpose are mountable to the universal sensor platform without, most preferably, much changing its weight or appearance or feel, which remains substantially that of a common hospital bracelet. For example, GPS monitors of the location of a patient wearer, sensors and monitors of patient/wearer physiology such as temperature and heartbeat, and still other medical sensors and alarms may commonly be mounted to, and made electrically compatible with, the limb band sensor platform of the present invention. The compact, durable and responsive medical (and veterinary) monitoring and alarming systems so constructed, mounted and connected are generally well accepted by the human and animal wearers because these wearable components are roughly equally as lightweight, compact, inconspicuous, non-irritating and unobtrusive as are hospital bracelets worn on the wrist. Indeed, the patient acceptance of these sensors is often improved by their integration with the limb band of the present invention, as next explained.

The purposes of the limb band sensor platform of the present invention are several. It serves to cost effectively provide a sophisticated universal and ubiquitous platform for (1) powering, (2) physically supporting, and (3) abetting communication with, diverse sensors and monitors that are mounted to the human body. The limb band sensor platform of the present invention may thus be subject to a “make versus buy” decision for a modern modular miniaturized wearable medical sensor system on the basis of its functionality alone. However, the bracelet universal sensor platform is also intended to accept new sensors, and to have a design, if not also a deployed, lifetime that exceeds that of designs, and the real-world implementations, of those sensors and monitors with which it is now, and in the future, deployed. The bracelet universal sensor platform relieves the designer(s) of a new sensor(s) or monitor(s) from worrying about any of (1) electrically powering the sensor or monitor, (2) recharging the power source of the sensor or monitor, (3) securely mounting the sensor or monitor to the body of a wearer, (4) providing an effective antenna for wireless communications and/or a plugjack for wired communications, and/or (5) according a path for the recovery, and re-use, of the sensor or monitor.

Equally importantly, however, in deciding to use the limb band sensor platform of the present invention of the present invention in a physiological sensor and/or monitoring system is that the limb band is intended to provide familiarity, comfort, and anonymity/privacy to patient wearers of the sensor devices. By use of the limb band the patient avoids wearing such a panoply of different, and differently positioned, sensors and monitors as to tend to induce any of confusion, embarrassment, dread, and/or loss of privacy (in that the nature of a sensor, and what condition it is monitoring, can often be visually easily determined in the prior art). Instead, the universal common sensor platform of the present invention accords a wearer patient a degree of dignity and discreteness, and does not make him or her to look like some heavily-wired escapee from some intensive care unit.

1. A Universal Band, or Bracelet, as a Universal Sensor Platform

Accordingly in one of its aspects the present invention is embodied in a wearable limb band including (1) a flexible strap body adaptable to wrap around the circumference of a limb of a human; (2) a rechargeable battery housed in the strap body; (3) a solar cell housed in the strap body converting light energy to electricity for recharging the battery; and (4) a platform area on the strap where a wearable sensor is mountable so as to be both physically transported and electrically connected to the battery.

The limb band preferably further includes an antenna within the strap body, where a sensor mountable at the platform area electrically connects to the antenna for wireless radio communication.

The limb band may, in certain embodiments, be considered to still further include a plugjack—mounted to the strap body, electrically connected to any sensor mounted at the platform, and presenting a plug to which a wired electrical signal connection can be made. This plugjack preferably supports both mechanical and electrical quick connection/dis-connection of any wired electrical signal connection.

In the most preferred he limb band the platform is in the topological shape of an annular ring (1) having and presenting within the body of the annulus both mechanical and electrical connections to a mounted and electrically connected sensor, and also (2) permitting this sensor when mounted to make unobstructed contact with the flesh of the human's limb through the central aperture to the annular ring.

Each of the flexible strap body, the rechargeable battery and the solar cell of the limb band are preferably water resistant and impervious to damage from normal wear upon the limb of the human.

The flexible strap body of the limb band is preferably adjustable in fit to the human limb to which it becomes attached.

At least one of the flexible strap body, the rechargeable battery and the solar cell preferably bear externally visible indicia identifying an individual wearing the bracelet.

Both the (1) rechargeable battery, and (20 the solar cell, of the limb band are preferably flexible with the flexible strap body of the limb band.

In use the limb band can mount an electronic sensor—affixed to the flexible strap body and powered by the battery—of the vitality of a person wearing the bracelet. For example, this electronic sensor may be a heartbeat sensor, or an optical pulse detector.

The limb band can still further mount a radio—affixed to the electronic sensor and powered by the battery—for reporting by radio the results of the vitality sensing.

Alternatively, the limb band can mount an electronic GPS sensor—affixed to the flexible band and powered by the battery—sensing the location of a person wearing the bracelet. In this case the preferred radio is affixed to the electronic GPS sensor and powered by the battery for wirelessly reporting by radio the results of the GPS sensing.

2. A Wearable Bracelet

In another of its aspects the present invention is embodied in a wearable bracelet platform for physiological sensing including (1) a flexible strap body adaptable to wrap around the circumference of a limb; (2) a battery housed within the strap and flexible therewith; and (3) a connector—mechanically attached to the strap body and electrically connected to the battery—suitable to both mechanically and electrically connect a wearable sensor. Upon such times as a sensor is connected and the bracelet is worn by an animal upon the animal's limb then the bracelet serves both to (1) mechanically adhere the sensor to the animal and to (2) electrically power the sensor.

The bracelet further preferably includes a plugjack for mechanically and electrically connecting a sensor. By operation of this plugjack upon such times as the sensor is plugged and the bracelet is worn by an animal then the bracelet serves both to mechanically adhere the sensor to the animal and to electrically power the sensor. The preferred plugjack preferably supports both mechanical and electrical quick connection and dis-connection.

In the preferred bracelet each of the a flexible strap body, rechargeable battery and solar cell are water resistant and impervious to use upon the human body. The flexible strap body is adjustable in fit to the limb.

In the preferred bracelet at least one of the flexible strap body, rechargeable battery and solar cell bear externally visible indicia identifying an individual wearing the bracelet.’

The bracelet may, and normally does, further include an electronic sensor, affixed to the flexible band and powered by the battery, of the vitality of a person wearing the bracelet. This electronic sensor of vitality may be, for example, a heartbeat sensor, and may further be an optical pulse detector.

The bracelet may further include a radio, affixed to the electronic sensor and powered by the battery, for reporting by radio the results of the vitality sensing.

In yet another embodiment, the bracelet may include a GPS sensor, affixed to the flexible band and powered by the battery, sensing the location of a person wearing the bracelet. In this case the bracelet preferably still further includes a radio, affixed to the electronic sensor and powered by the battery, for reporting by radio the results of the GPS sensing.

These and other aspects and attributes of the present invention will become increasingly clear upon reference to the following drawings and accompanying specification.

### BRIEF DESCRIPTION OF THE DRAWINGS

FIG. 1 is a perspective view of a preferred embodiment of a limb band sensor platform in accordance with the present invention, including some variants in the mounted sensor and its attachment(s).

FIG. 2, consisting of FIGS. 2a through 2f, are plan views of various portions of the flexible band, and it contained components, of the limb band sensor platform of the present invention previously seen in FIG. 1.

FIG. 3, consisting of FIGS. 3a and 3c, are respective exterior, side, and interior plan views of the flexible band of the limb band sensor of the present invention, with a flexible phot-voltaic cell particularly shown in FIG. 3a and with a flexible RF antenna shown in X-ray view in FIG. 3c.

FIG. 4, consisting of FIG. 4, are plan views of the interface pocket of the limb band sensor respectively at normal and magnified views, and of the definitions of the contacts to a mounted sensor.

FIG. 5 is an exploded perspective view of one preferred embodiment of the limb band sensor platform, including preferred components, of the present invention.

FIG. 6, consisting of FIGS. 6a though 6f, are detail perspective and plan views of both the mounting pad of the limb band sensor platform of the present invention, and of sensors and connectors and the like that connect at, and to, said mounting pad.

### DESCRIPTION OF THE PREFERRED EMBODIMENT

1. Problems Addressed by the Limb Band Sensor Platform of the Present Invention

As well as a general requirement for portable/wearable physiological sensors, there is in particular a present, circa 2011, inability for hospitals and caretakers to properly track Alzheimer's, autistic, and patients with neurological disorders. The problem extends to any and all persons admitted to medical care facilities who have the potential for real emergencies to take place, in that given any particular emergency could potentially go unattended because the person cannot effectively communicate his/her incipient needs. Such a person needs a vitality monitoring and alarming device. Such a device can also solve similar problems in distressed animals and pets of all sorts, including tracking such pets when there is a confinement issue.

Present (circa 2012) patient-wearable sensing and monitoring devices are in general not self charging, and may run out of power leaving the patient vulnerable.

The limb band sensor platform of the present invention uses photovoltaic technology to recharge a battery. In (1) normal use and lighting conditions, including indoors, to power (2) one or two typically (radio-communicating) microelectronic sensors, the limb band has no need for AC power recharging. Additionally, the band interchangeably can mount a number of sensors—including some two or more independently operating sensors—at a single time. The wearable limb band can be interchanged with a number of diverse sensors for the purpose of monitoring patients/pets with a variety of specific needs. The preferred limb band also has a antenna that connects to a radio-communicating sensor or monitor not only for permitting a physically-mounted and electrically-connected sensor/monitor to broadcast a string radio signal reporting the condition of the wearer, but also for strongly receiving such radio signals as do potentially also permit a patient to communicate with his/her caretaker. A patient who is lost or confused can take comfort in hearing a familiar voice, and this can occur though a same module that serves as a primary sensor/monitor, or through an entirely different module simultaneously mounted to the limb band (or even to another limb band simultaneously worn). The reason that the transmitted, and received, radio signals are “strong” is simply that the limb band is commonly physically much larger than the microelectronic sensor/monitors that it serves to mount and connect, and, since the antenna will be seen to be substantially coextensive with the entire circumference of the limb band, the relatively large antenna of the limb band serves as a more efficient and effective radio radiator or receiver than would any smaller radio antenna built within the sensor or monitor.

Because of its modularity, the system of the present invention based on a limb band sensor platform clearly accords flexibility and cost effectiveness in addressing diverse patient conditions and problems.

For example, when the band of the present invention mounts a GPS sensor then it can act to solve the problem of run-away Alzheimer's patients, patients with autism, cardiac problems, sleep apnea, epileptics or other health problems. It does so by monitoring and locating these patients, who often need constant attention.

Additionally, with one or more appropriate sensors, the band of the present invention can serve monitor the vitality and location of patients/animals around the clock. It conveniently solves that problem of constantly having to be charged by its self-charging apparatus. In general the limb band with appropriate sensors and monitors serves to solve diverse patient/animal location challenges and health challenges by transmitting information to a remote location such as a cell phone, or a lap top or any electronic device that can receive and interpret a signal.

Still further additionally the limb strap of the present invention can be used as an anti-drowning device if an appropriate sensor is mounted. It also has the ability to be used for military applications, specifically to be used as a device to let commanding officers know of the whereabouts of their enlisted men/women. A proper system will both give location coordinates and offer the wearer the ability to communicate his/her physical condition to command posts via a both (1) a vitality sensor, or module, and (2) a GPS/Satellite technology geospatial positional sensing/monitoring module. The military would benefit from a self-powering device with the capability to track individuals and communicate pertinent information.

Yet still further the limb band sensor platform can mount a vitality monitor for children or any patient—especially in a dental office, podiatrists office, or any outpatient facility where unique vitality monitoring is needed. A system based on the limb band sensor platform of the present invention can be adapted to fit all these unique uses, and more.

2. Purposes of the Limb Band Sensor Platform of the Present Invention

As stated above, inability for hospitals and caretakers to properly track Alzheimer's, autistic, and patients with neurological disorders can be troublesome. The problem extends to any and all admittance to medical care facilities where exists a potential for real emergencies to take place, given that any particular emergency might go unattended save for a vitality monitoring and alarming device. As was also stated above, a vitality monitoring and alarming device could also serve to solve similar problems in distressed animals and pets of all sorts, including tracking such pets when there is an elopement issue. The present invention of a limb band sensor platform helps to solve these problems.

This invention provides a band (1) that is mechanically secured about a limb, normally the wrist (ergo, a wristband), and (2) that provides a long-lasting and self-sustaining power source to any wireless communication device mounted to the band, or (with wired connection) close by on the limb (the wrist) of the patient (or the collar of a pet). The band of the present invention can also serve as a collar for pets where it again provides a long-lasting and self-sustaining power-source for a sensor and/or monitor of the pet's for their vitality of position,. The band or collar is water resistant and tamper proof. An onboard battery is charged/re-charged when an onboard solar cell is exposed to light illumination, including indoor ambient lighting.

Both the battery and the solar cell are normally flexible with the band. The circumference of the band is variably adjustable to fit different sizes of (1) limbs, including any of (2) wrists and (3) ankles and (4) necks.

The preferred limb band is able to mount one or more communication modules or like electrical circuits each of which takes, most typically, up to 3.7 v.d.c. from the battery.

So constructed and used the limb band sensor platform of the present invention differs from all structures know to the inventors, being that there are no structures known to the inventors that combine all of the principle elements of the limb band of the present invention. For example, although wearable sensors presently exist, these are commonly presently tied to electrical power sources that require intermittent charging by direct connection to an external power source. Other wearable sensor systems are non-diversified, and lack ability to monitor and/or track patients having a multitude of differing specific needs.

Present systems know to the inventors are additionally limited by their inability to be self-sustaining in terms of their power source(s). Present systems are not adaptable in terms of the patients that they are monitoring. For example, present systems do not have the ability to provide different sensing and/or monitoring services to a cardiac patient and an Alzheimer's patient from a common platform, whereas the limb band of the present invention has that and ours that capability when an appropriate electronic sensor/monitor is mounted to the band. The mounted sensor/monitor can even have the ability to scan its information onto a computer or cell phone via temporary wired connection, or wirelessly via radio.

In use the limb band sensor platform of the present invention can mount a vitality monitor for children, or any patient—especially in a dental office, podiatrist's office, or any outpatient facility where unique vitality monitoring is needed. Whenever there is a dearth of skilled professionals—whether dentists, podiatrists, caretakers or like personnel—to adequately monitor their patients, the sensors and monitors universally supported by limb band sensor platform of the present invention can come into play.

The preferred version of the limb band sensor system preferably includes any of (1) a flexible strap, with (2) a clasp mechanism for adjustably affixing the strap about a limb, (3) one or more housings and/or mounting pads for accepting a sensor, a monitor and/or like electrical circuits, (4) metal shims as necessary, (5) a flexible battery, (6) twisted buss-wire for interconnection, and/or (7) photovoltaic (solar) cells, as hereinafter shown and described.

In greater detail, the strap, band or collar is preferably molded in a special plastic-rubber resin which will withstand sweat, vibration, water resistant.

The clasping mechanism preferably consists of the spaced-apart holes and plastic finger-squeezable plastic rivets of a common hospital-type ID band. The band is adjustable by those holes that are selectively mated by the rivet(s) so as to adjustable to fit any size. Once secured in place, it will not come off unless it's removed with proper plier tools, or cut off with scissors. The flexible rubber of plastic band material will not hurt the pet or people as it doesn't have any sharp edges.

An internal cavity within the band may house and protect the battery and any associated or electrical circuitry. A thin metallic shim may also be used to protect and to electrically connect the battery. The battery itself is selected not only so as to be able to power a range of sensors and monitors that a selectively mountable to the band, but is flexible so as to accommodate comfortable fitting on patients, people or pets.

The band can house twisted buss-wire for electrical connection of components. This buss wire also precludes that the band should be easily cut it regions where it exists

The preferred rubber-resin material of the band is readily shaped and molded to provide protective covering to the battery, shim, and buss-wire.

The photovoltaic, or solar, cells and vibration cells are preferably infused and housed into the plastic wrist band or collar molding. These cells of course serve to give the limb band senor platform its self-powering ability. Under normal illumination these photovoltaic, or solar, cells will generate adequate energy so as to charge the battery to provide constant power to any common microelectronic mounted sensor(s), monitor(s) and/or like electrical circuit(s).

3. How the Limb Band Sensor Platform of the Present Invention Preferably Works

An electronic sensor or monitor mounted to the limb band sensor platform of the present invention has the ability to receive information from a subject—whether human or animal—and, most preferably, to transmit and communicate said information in near real time to remote locations, for example to cell phones, lap tops, or other electronic devices. The electronic sensors get the power to do this from the battery. The battery is charged and re-charged by the photovoltaic, or solar, cell(s). There can optionally be included an alert switch within the band which will be triggered when the band is opened for any reason other than the legitimate reasons of an the administrator, with this alert electronically communicated in cooperation with a sensor or monitor module mounted upon the band.

4. Details of the Preferred Embodiments of a Limb Band Sensor Platform in Accordance with the Present Invention

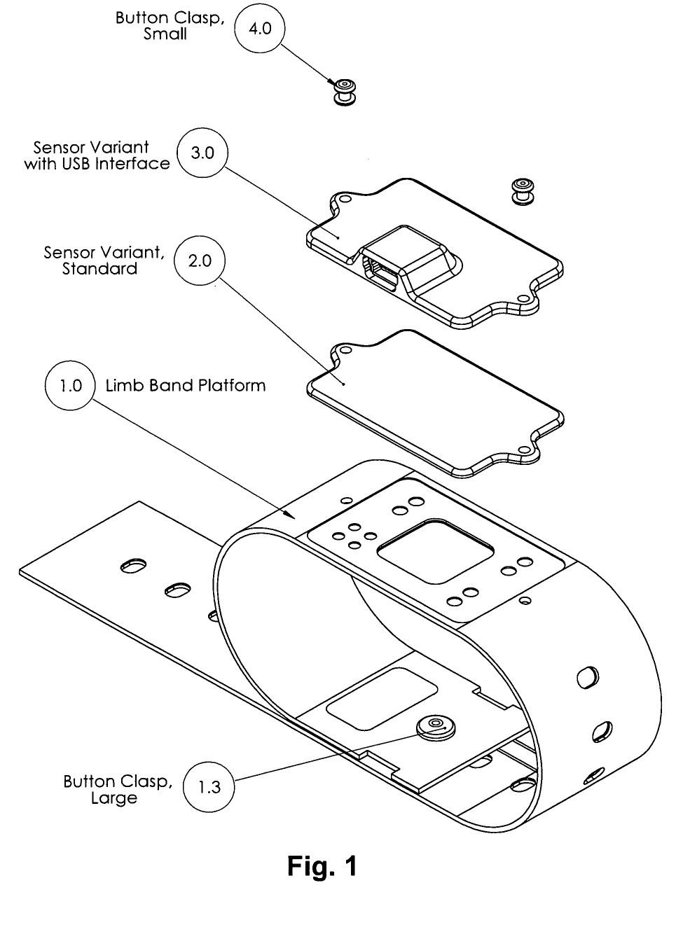

An exploded perspective view of a preferred embodiment of a limb band sensor platform 1.0 in accordance with the present invention, including some variants in the mounted sensor 2.0 and its attachment(s) 3.0 is shown in FIG. 1.

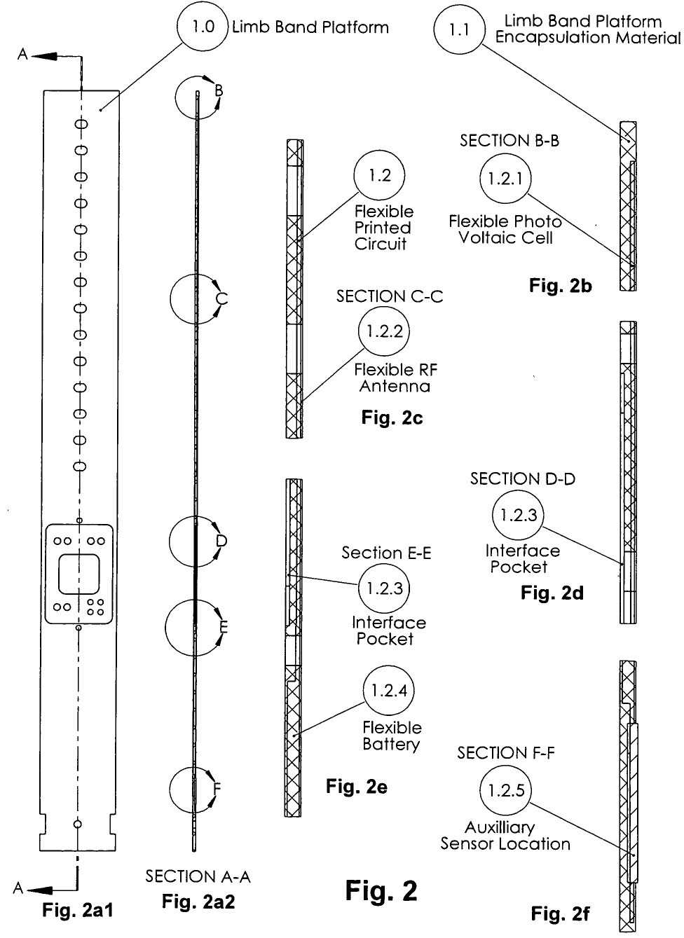

The Limb Band Platform 1.0 is secured to a human limb (not shown) by button clasp, large 1.3. A sensor variant. standard 2.0 is physically attached to the limb band platform 1.0 by button clasps 4.0. The same sensor variant. standard 2.0 is electrically connected, to the limb band platform 1.0 by pads 2.2-2.11 (seen in FIG. 2).

Optional sensor variant with USB interface 3.0 likewise physically and electrically attaches to limb band platform 1.0. This sensor 3.0 may further attach to sensor 2.0, and vice versa.

All sensors 2.0, 3.0 physically attach to the limb band platform 1.0 by button clasp, small, 4.0. In some variant embodiments the sensor variant with USB interface 3.0 is supplied by the manufacturer of the limb band platform 1.0, and is considered a component part thereof. There needs be no active “sensor” within “sensor variant with USN interface 3.0”, and this add-on module may be used simply to provide a USB port to sensor variant, standard 2.0.

Plan views of various portions of the flexible band 1.2, and it contained components, of the limb band sensor platform 1.0 of the present invention (previously seen in FIG. 1) are shown in FIGS. 2a1, 2a2, and 2b through 2g.

The flexible band 1.2 of the limb band sensor platform 1.0 is shown in top plan view in FIG. 2a1, and again in side plan view (taken along aspect line A-A of FIG. 2a1) in FIG. 2a2. The material of the limb band sensor platform 1.0, and of its flexible band 1.2, is predominantly plastic.

A flexible photo voltaic cell 1.2.1 is shown in FIG. 2b, which FIG. 2b is taken along aspect line B-B of FIG. 2a2.

A flexible printed circuit 1.2 and a flexible RF antenna 1.2.1, are shown in FIG. 2c, which FIG. 2c is taken along aspect line C-C of FIG. 2a2.

An interface pocket 1.2.3 and a flexible battery 1.2.4 are shown in FIG. 2d, which FIG. 2d is taken along aspect line D-D of FIG. 2a.

The same interface pocket 1.2.3 and a flexible battery 1.2.4 are shown in FIG. 2e, which FIG. 2e is taken along aspect line E-E of FIG. 2a2.

An auxiliary sensor location 1.2.5 is shown in FIG. 2f, which FIG. 2f is taken along aspect line F-F of FIG. 2a.

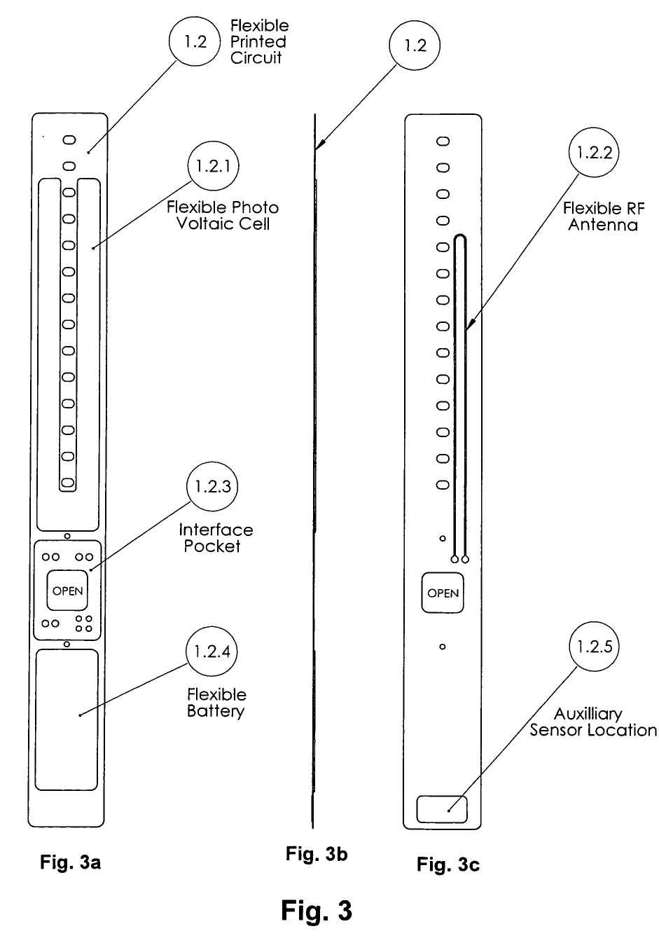

FIGS. 3a, 3b and 3c are further plan views of the flexible printed circuit (band) 1.2 previously seen in FIG. 2c. FIG. 3a shows a top plan view of a first layer of flexible printed circuit 1.2 showing each of s photo voltaic cell 1.2.1, an interface pocket 1.2.3, and a flexible battery 1.2.4. Electrical connections between these elements are trivial, and within the skill of a practitioner of the electrical engineering arts.

FIG. 3b shows a side plan view of flexible printed circuit 1.2.

FIG. 3c shows a bottom plan view of a bottom, lowermost, layer of the flexible photo printed circuit 1.2, now showing the flexible RF antenna 1.2 and an auxiliary sensor location 1.2.5, This auxiliary sensor location 1.2.5 provides yet another location where a sensor or monitor may physically mounted and electrically connected, as opposed to riding piggyback (as shown in FIG. 1). The “OPEN” space in flexible photo printed circuit 1.2 permits mounted sensors to contact the skin of the wearer of the limb band platform 1.0 of the present invention.

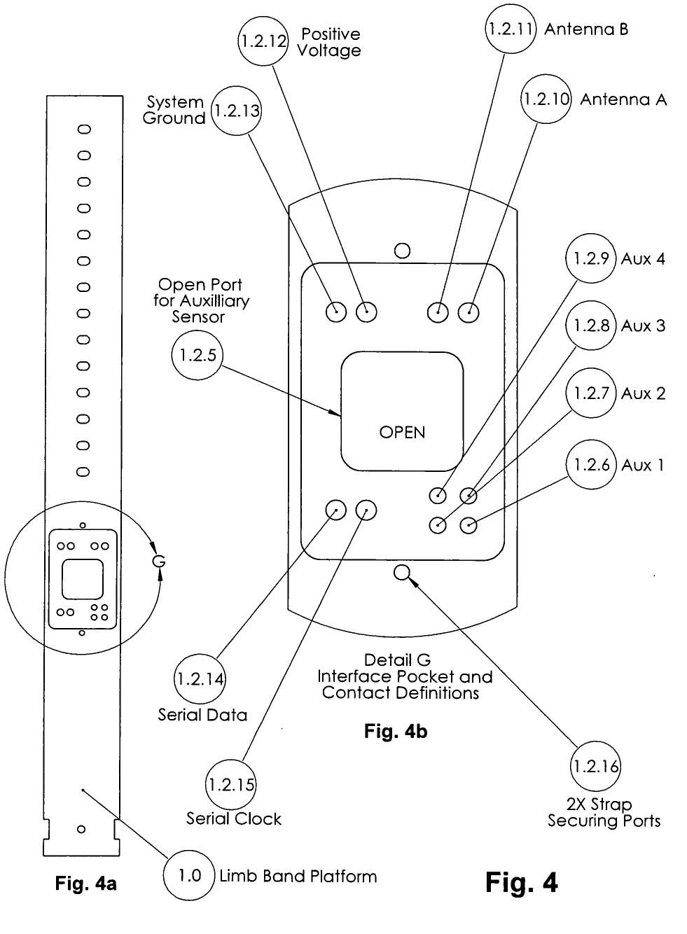

FIG. 4a shows, in detail plan view taken about aspect line G-G appearing in FIG. 4a, the interface pocket and contact definitions of the interface pocket 1.2.3 (previously seen in FIG. 3a. An open port for auxiliary sensor 1.2.5, aux 1 element 1.2.6, aux 2 element 1.2.7, aux 3 element 1.2.8, aux 4 element 1.2.9, antenna A 1.2.10, antenna B 1.2.11, positive voltage 1.2.12, system ground 1.2.13, serial data 1.2.14, serial clock 1.2.15 and strap securing ports 1.2.16 are shown,

An exploded perspective view particularly more particularly showing the flexible battery 1.2.4, and the photovoltaic cell 1.2.1 (both previously seen in FIG. 2), of the limb band sensor platform 1 of the present invention is shown in FIG. 4. Flexible Printed Circuit 1.2 includes Flexible Photo Voltaic Cell 1.2.1, Interface Pocket 1.2.3 and Flexible Battery 1.2.4.

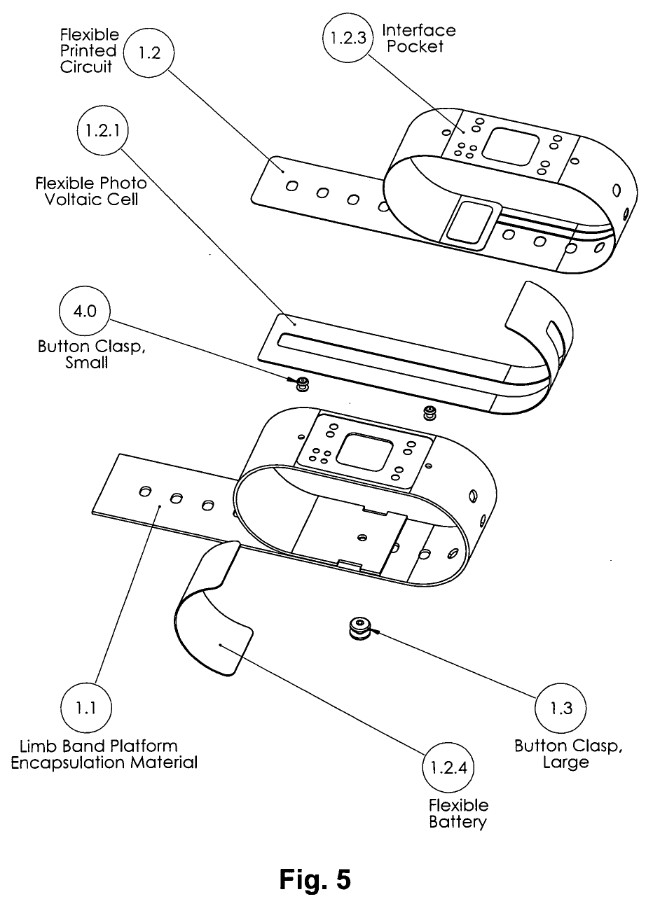

An X-ray plan view particularly showing the antenna within the limb band sensor platform 1 of the present invention is shown in FIG. 5. Flexible RF Antenna 1.2.2 and Auxilliary Sensor Location 1.2.5.

An exploded perspective view of one preferred embodiment of the limb band sensor platform 1, including preferred components, of the present invention is shown in FIG. 5. This FIG. 5 shows the flexible printed circuit 1.2 featuring the flexible photo voltaic Cell 1.2.1 and the interface pocket 1.2.3. The limb band platform encapsulation material 1.1 is secured by button clasp, large 1.3 while an sensor(s) is (are) [not shown in FIG. 5] secured by button clasp, small 4.0. Also shown are the flexible printed circuit 1.2 and the flexible battery 1.2.4,

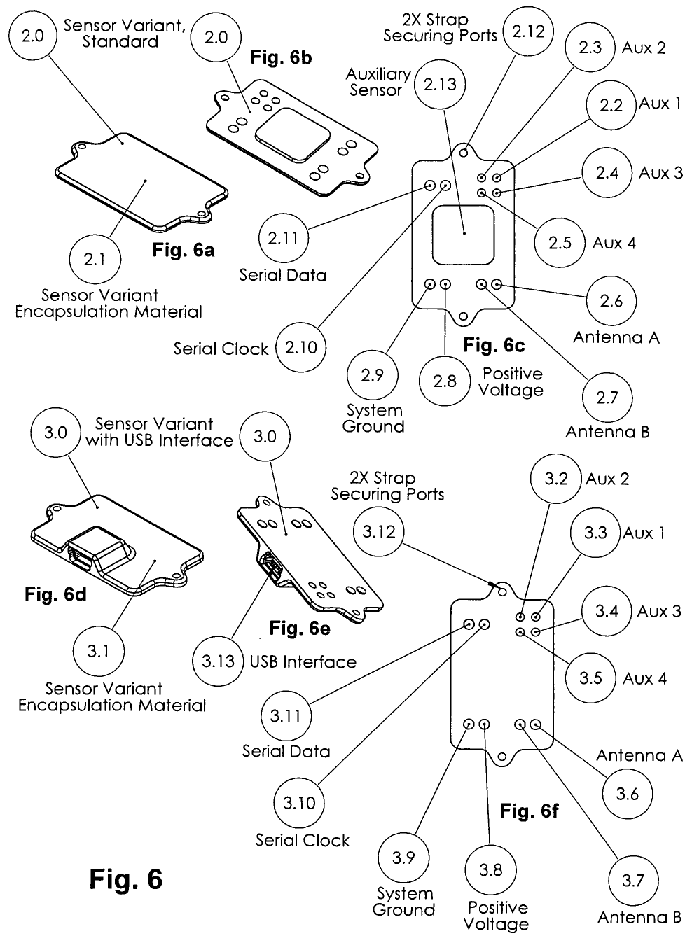

Detail perspective and plan views of both the mounting pad of the limb band sensor platform of the present invention, and of sensors and connectors and the like that connect at, and to, said mounting pad are shown in FIG. 6, consisting of FIGS. 6a though 6f.

Sensor variant, standard 2.0 of FIG. 6a incorporates sensor variant encapsulation material 2.1.

The interface pocket 1.2.3 of the of the flexible printed circuit 2.0 of the limb band platform 1.0 of the present invention is shown in detail in FIG. 6c. This interface pocket 1.2.3 has an aux 1 element 2.2, aux 2 element 2.3, aux 3 element 2.4, and aux 4 element 2.5; an antenna A 2.6 and an antenna B 2.7; a positive voltage 2.8 and a system ground 2.9; a serial Clock 2.10 and serial data 2.11. Physically, (2×) strap securing ports 2.12 secure auxiliary sensor 2.13.

The sensor variant with USB Interface 3.0 previously seen in FIG. 1 is now again shown in FIG. 6d. It is made using, among other parts, sensor variant encapsulation material 3.1

The flip side of this same sensor variant with USB interface 3.0 is shown in FIG. 6e.

Finally, the flip side of the interface pocket 1.2.3 of the of the flexible printed circuit 2.0 of the limb band platform 1.0 of the present invention—previously shown in FIGS. 6b and 6c—is shown again in FIG. 6f. The elements of this FIG. 6f are exactly as previously identified in FIG. 6c.

As well as serving as a sensor platform, the limb band platform 1.0 of the present invention can present the same, or like, identification indicia as does a standard hospital wrist identification bracelet.

According to these variations, and still others within the skill of a practitioner of the medical appliance design arts, the present invention should be considered in accordance with the following claims, only, and not solely on accordance with those embodiments within which the invention has been taught.

What is claimed is:

1. A wearable limb band comprising:
a flexible strap body adaptable to wrap around the circumference of a limb of a human;
a rechargeable battery housed in the strap body;
a solar cell housed in the strap body converting light energy to electricity for recharging the battery; and
a platform area on the strap where a wearable sensor is mountable so as to be both physically transported and electrically connected to the battery.

2. The limb band according to claim 1 further comprising:
an antenna within the strap body;
wherein a sensor mountable at the platform area electrically connects to the antenna for wireless radio communication.

3. The limb band according to claim 1 wherein the platform further comprises:
a plugjack mounted to the strap body, electrically connected to any sensor mounted at the platform, and presenting a plug to which a wired electrical signal connection can be made.

4. The lib band according to claim 3
wherein the plugjack supports both mechanical and electrical quick connection/dis-connection of any wired electrical signal connection.

5. The limb band according to claim 1
wherein the platform is in the topological shape of an annular ring having and presenting within the body of the annulus both mechanical and electrical connections to a mounted and electrically connected sensor, and also permitting this sensor when mounted to make unobstructed contact with the flesh of the human's limb through the central aperture to the annular ring.

6. The limb band according to claim 1
wherein each of the flexible strap body, the rechargeable battery and the solar cell are water resistant and impervious to damage from wear upon the limb of the human.

7. The limb band according to claim 1
wherein the a flexible strap body is adjustable in fit to the limb.

8. The limb band according to claim 1
wherein at least one of the flexible strap body, rechargeable battery and solar cell bear externally visible indicia identifying an individual wearing the bracelet.’

9. The limb band according to claim 1
wherein rechargeable battery is flexible with the flexible strap body.

10. The limb band according to claim 1
wherein solar cell is flexible with the flexible strap body.

11. The limb band according to claim 1 further comprising:
an electronic sensor, affixed to the flexible strap body and powered by the battery, of the vitality of a person wearing the bracelet.

12. The limb band according to claim 11 wherein the electronic sensor of vitality comprises:
a heartbeat sensor.

13. The limb band according to claim 13 wherein the electronic heartbeat sensor of vitality comprises:
an optical pulse detector.

14. The limb band according to claim 11 further comprising:
a radio, affixed to the electronic sensor and powered by the battery, for reporting by radio the results of the vitality sensing.

15. The limb band according to claim 1 further comprising:
an electronic GPS sensor, affixed to the flexible band and powered by the battery, sensing the location of a person wearing the bracelet.

16. The bracelet according to claim 15 further comprising:
a radio, affixed to the electronic GPS sensor and powered by the battery, for wirelessly reporting by radio the results of the GPS sensing.

17. A wearable bracelet comprising:
a flexible strap body adaptable to wrap around the circumference of a limb;
a battery housed within the strap and flexible therewith; and
a connector, mechanically attached to the strap body and electrically connected to the battery, suitable to both mechanically and electrically connect a wearable sensor;
wherein upon such times as a sensor is connected and the bracelet is worn by an animal upon the animal's limb then the bracelet serves both to (1) mechanically adhere the sensor to the animal and to (2) electrically power the sensor.
